(19) United States
(12) Patent Application Publication
(10) Pub. No.: US 2007/0067045 A1
(43) Pub. Date: Mar. 22, 2007

(54) Systems and methods for skin wrinkle removal

(75) Inventors: Loc Phan, San Jose, CA (US); Amir Abolfathi, Woodside, CA (US)

Correspondence Address: TRAN & ASSOCIATES, 6768 MEADOW VISTA CT., SAN JOSE, CA, 95135, US

(73) Assignee: AR2 Partners, Inc.

(21) Appl. No.: 11/230,234

(22) Filed: Sep. 19, 2005

(51) Int. Cl. A61F 2/02

(52) U.S. Cl. 623/23.72; 424/424

(57) ABSTRACT

An implant is disclosed that reduces wrinkles. The implant may be in the shape of a cylinder with a constant or a varying cross-section and length. The implant contains a gel that has limited flow capability. The implant can also be a balloon that may or may not have multiple compartments. The compartments of the balloon maybe filled with saline, radio contrast agents, or polymeric gel. The gel can contain or not contain radio contrast agents for subsequent imaging of the implant for medical purposes.

### BACKGROUND

In the aging process, various signs appear on the skin resulting from a modification of skin structure and of cutaneous function. The main clinical signs of aging are, among others, the appearance of fine wrinkles and deep wrinkles. These signs of aging increase with age. A disorganization of skin “texture” is more particularly observed, meaning that micro-contours are less regular and show an anisotropic character.

In young skin, the collagen just beneath the surface of the skin forms an organized lattice with good elasticity and flexibility. During aging, the collagen changes its structure impacting negatively on the cosmetic appearance of the skin.

As noted in U.S. Pat. No. 6,896,889, it is known to treat these signs of aging using cosmetic or dermatological compositions containing actives adapted to fight against such signs of aging, such as α-hydroxy-acids, β-hydroxy-acids and retinoids. Such actives act on wrinkles primarily by eliminating dead skin cells and accelerating the cell renewal process. However, the visible effect of such compositions is generally observed after some application time, ranging from a few days to several weeks. The '689 patent uses mineral fillers in a composition provides such a composition, after application onto the skin, with a smoothing effect of the skin surface layer when such fillers are in the form of colloid particles in a stable dispersion in an aqueous medium. Such fillers in the form of colloid particles can therefore be used as tensing agents in immediate effect anti-wrinkle compositions. The compositions includes at least one mineral filler, with the exclusion of any mixed silicate, characterized in that the mineral filler is in the form of colloid particles so that at least 70% of them have a diameter in the range from 0.1 to 100 nm, preferably from 3 to 30 nm, in a dispersion in an aqueous, alcoholic or hydro-alcoholic medium.

In addition to compositions such as fillers, lasers have been used for wrinkle removal as discussed in U.S. Pat. No. 6,881,212. Conventional laser wrinkle removal techniques select a wavelength so that the laser energy is highly absorbed in water, the current lasers of choice being the C02 laser at 10.6 um wavelength and the Erbium YAG laser at 2.94 um wavelength. In this non-selective process, pulses of laser energy are applied to the skin surface, each pulse vaporizing a layer of tissue between 30 to 60 um in thickness. Normally, the first pass of the laser removes a thin layer of the epidermis without damaging the basal layer. Successive passes over the same area penetrate into the dermis and heat the collagen. The laser operator sees this thermal build-up “shrink” the skin in “real time” , tightening up the skin's appearance. When the desired clinical outcome is achieved, the operator ceases applying laser pulses. It is therefore apparent that the quality of the cosmetic result is highly dependent upon the experience and skill of the operator. In the case of C02 laser wrinkle removal, post-treatment supervision of the patient is a necessity. Immediately after treatment, the skin is essentially an open wound requiring dressings in place for 2-10 days. Additionally, topically applied lotions are required for patient comfort and prevention of infection. Post-operative infection is common, primarily due to removal of the natural protective barrier of the skin, with a reported incidence of between 4.5 to 7%.

On average, with C02 laser wrinkle removal, post-treatment erythematic is present for 4-5 months. This compares to 2-3 months following a Chemical Peel. Also, the incidence of side effects is significant, the most common being hyper pigmentation occurring in 30-40% of cases. Higher incidences are reported in darker skin types. A delayed hypo pigmentation, which can occur up to a year after the procedure was performed, has recently emerged as a complication of aggressive laser resurfacing.

On a parallel note, U.S. Pat. No. 6,847,848 discloses a minimally-invasive treatment of diseased sub-cutaneous tissue of a patient through an integrated-structure inflatable balloon catheter design that includes a longitudinal structure having a sharply-pointed insertion needle at a distal end of the longitudinal structure and an inflatable balloon situated intermediate a proximate end and the distal end of the longitudinal structure which is attached to said longitudinal structure. With the inflatable balloon in a deflated state, the insertion needle may be used to puncture the patient's skin and underlying sub-cutaneous tissue and place the deflated balloon in proximity to the diseased sub-cutaneous tissue, The balloon is then inflated to press against and thereby spatially deform the diseased sub-cutaneous tissue, after which the deformed diseased sub-cutaneous tissue may be therapeutically heated. This heating may be sufficient to cause the creation of a permanent cavity in the deformed diseased sub-cutaneous tissue which persists after the catheter is withdrawn. This permits any selected one of various therapeutic substances to be introduced into this a permanent cavity.

### SUMMARY

An implant is disclosed that reduces wrinkles. The implant may be in the shape of a cylinder with a constant or a varying cross-section and length. The implant contains a gel that has limited flow capability. The implant can also be a balloon that may or may not have multiple compartments. The compartments of the balloon maybe filled with saline, radio contrast agents, or polymeric gel. The gel can contain or not contain radio contrast agents for subsequent imaging of the implant for medical purposes.

Advantages of the implant system may include one or more of the following. The system allows for skin smoothing and skin wrinkle and fine wrinkle attenuation. The system provides a naturally smooth skin without requiring complex laser surgery or chemical intervention. The desired effect can be achieved quickly and with minimally invasive surgery. The risk of infection can be reduced due to the minimally invasive therapy. The gel material that filled the balloon is compliant at different frequencies with the surrounding tissue so that it matches the response of the tissue to the exterior touches, thus creating the natural feelings to touches at the site. The compliant nature of the gel filler makes the expression of the patient to appear more natural then other procedures by not distorting the natural the facial contours. Historical photographs can be used to tune the level augmentation so that the treated areas will blend in with other untreated areas by the gradations of treatment and compliant level. The filler material, being a higher molecular gel, allows a semi-porous balloon material to be used which allows continuous zero order drug delivery method, the most desirable controlled delivery method. The system can be used for delivery of anti-aging compounds to better control the aging of surrounding tissue. It can also be used for delivery of birth-control pharmaceuticals. The semi-porous nature makes transport of nutrients across the implant more easily making the device much bio-compatible than others that cannot and would cause necrosis to immediately surrounding tissue. The device is easily removable for additional drug loading.

### BRIEF DESCRIPTION OF THE DRAWINGS

The present invention is illustrated by way of example, and not by way of limitation, in the figures of the accompanying drawings and in which like reference numerals refer to similar elements and in which:

FIG. 1 shows an exemplary implant or balloon.

FIG. 2 shows an exemplary Implant Catheter Being Inserted.

FIG. 3 shows the Implant Catheter of FIG. 2 Being Removed

FIG. 4 shows the implant as deployed and compliant with the tissue.

### DESCRIPTION

As a preface to the detailed description, it should be noted that, as used in this specification and the appended claims, the singular forms “a”, “an”, and “the” include plural referents, unless the context clearly dictates otherwise. All percentages (%) listed for gas constituents are % by volume, unless otherwise indicated.

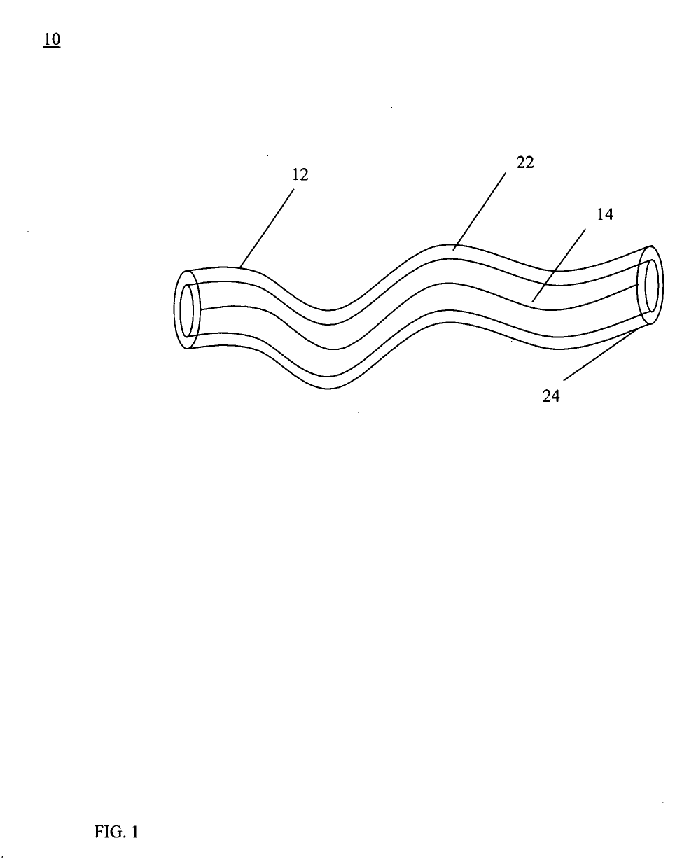

FIG. 1 shows a first embodiment of an implant 10 to reduce wrinkles. The implant may be in the shape of a cylinder 12 with constant or varying cross-section and length. The implant contains a gel 14 that has limited flow capability. The implant can also be a balloon that may or may not have multiple compartments. The compartments of the balloon maybe filled with saline, radio contrast agents, or polymeric gel. The gel can contain or not contain radio contrast agents for subsequent imaging of the implant for medical purposes.

In one embodiment, an implantable biocompatible gel 22 is encapsulated within an elastomeric tube or shell 20. Another tube or shell 24 is use to cover the inner shell 20 and to form a double bag. The inner layer or shell 20 is filled with a customized viscous gel. The outer shell 24 keeps the gel 22 from incorporating or adhering to the tissue for later removal if needed. In another embodiment, a hydrogel with customized rheological properties is used to allow the ability to alter the physical appearance of the implant as needed. Examples of gel formulations are:
A. 99% PEG (1000 MW, the 1000 signifies the molecular weight of the ethylene oxide repeating units CH₂CH₂O, and may vary in the number of repeating units to make adjustments in viscosity) cross-linked polymer, the cross-link density is 0.01-1% by wt/wt basis. Changing the cross-linking density can also change the viscosity; however, this method is sensitive and hard to control. The cross-linking compound may be 1,4-butanedioldimethacrylate, 1,6-hexanedioldimethacrylate, allylacrylate, multifunctional monomers. The mixture can be initiated with 0.1% wt/wt peroxide, Perkadox, azobisisobutyl nitrile and heat or an ultra violet light source.
B. 90% 2-hydroxy ethylmethacrylate, 9% butylmethacrylate, 0.1%-1% multifunctional monomers, thermally sensitive initiators or UV sensitive initiator benzoin methyl ether. This composition does not pick up as much water as the A composition from above.
C. Polyvinyl alcohol (PVA) is dissolved in water to create gel. The viscosity depends on the concentration of the dissolved PVA. An example is 10% Mowiol in 90% water, wt/wt, will make a solution with viscosity similar W30 motor oil.
D. Ninety percent (90%) hydroxy-terminated vinyl silicone, 9% N-vinyl pyrolidone, 0.1%-1% thionyl chloride, platinum catalyst. The mixture is polymerized in an oven at 100 C. for 2 hours. The resulting gel has very good viscoelastic property.

In yet another embodiment, the gel 22 can include small particles of radio opaque materials such as bio-compatible metals such as gold and platinum or metal salts and oxides such as barium sulfate are added for better visualization of the implants and treatment assessment. Exemplary radio opaque gel 22 may include:
A. A 5%-40% barium sulfate very small particle powder, wt/wt, is dispersed into any of the gel formulations above will impart varying level of radio opacity. Usually a 40% has opacity level equals to that of high-density metals like silver or gold.
B. Other high-density salts may also be used to impart radio opacity in the same wt/wt level such as bismuth sulfate. The gels compositions above with 5%-40% bismuth sulfate added after the polymerization has completed. Examples A and B are of salts of heavy atoms, such as barium and bismuth. The same may be done with other heavy atoms.
C. Oxides of heavy atoms may also be used in a similar manner. Some examples are zirconium dioxide, silver oxides, platinum oxides, and tungsten oxides.
D. An example of a gel formulation with barium sulfate: 80% polyethylene glycol (1000) methacrylate, 10% butyl methacrylate, 9% methylmethacrylate, 0.9% polyethylene glycol (550) dioldimethacrylate, wt/wt. The barium sulfate is added to the monomer composition at 20% BaSO4 to 80% monomer.

In yet another embodiment, an anti-inflammatory drug coating can be applied to the outer shell 24 to minimize the foreign body response by the body to the implant. Exemplary drugs can include dexamethasone sodium acetate and other similar drugs used widely in pace makers and defibrillator leads system. An example list of drugs is as follows:
1. Anti-inflammatory (arthrotec, asacol, auralgan, azulfidine, bextra, celestone, daypro, deltasone, diclofenac, etodolac, indocin, ketoprofen, iodine, mobic, nabumetone, naproxen, piroxicam, ponstan, prednisone, rofecoxib, salofalk, solumedrol).
2. Antibiotics (Amlodipine, Besylate, Amoxicillin, Amoxil, Amphotericin, Ampicillin, Augmentin, Avelox, Bactrim, Bactroban, Biaxin, Ceftriaxone, Cefzil, Cephalexin, Chloramphenicol, Cipro XR, Clostebol, Cloxacillin, Cotrim, Daraprim, Dicloxacillin, Doxycycline, Eryacne, Erythromycin, Ethambutol)
3. Anti-aging compounds
4. Anti-oxidants

In one embodiment, one or more of these drugs may be incorporated into gel formulation to affect controlled delivery. The level of control over the delivery of the drug depends on the interaction between the specific drug and the polymer. The interactions are usually at the functional group level. The properties that affect the controls on the delivery are solubility, diffusion, and permeability between the polymer and the drug or drugs.

In yet another embodiment, a biocompatible polymer with a modulus that matches the modulus of the surrounding tissue may be used for natural feel and appearance. The polymer is a hydrogel that will absorb a preset amount of water will be used to control the specific desired modulus. To control the shape of the polymer, the outer surface maybe lightly cross-linked so that it will not flow into locations that are not desired. The cross-linking is gradient and light enough so that it will more elastomeric.

In a further embodiment, a balloon catheter is used to fill the deep wrinkle. The balloon is long and extremely flexible, much more than normally seen in vascular system catheter. The balloon can be 1/5 the flexibility of the catheter for compliant against the skin surface. To stiffen the catheter, coaxial strength, for insertion requires a guide wire. Once the catheter is in place, the guide wire is retracted to give the catheter back its extreme flexibility and compliant. However, before retracting the guide wire, a saline solution is injected into the balloon, just enough volume to fill the wrinkle skin, making it smooth. The gel may already be loaded inside the balloon and absorbs water to fill the balloon.

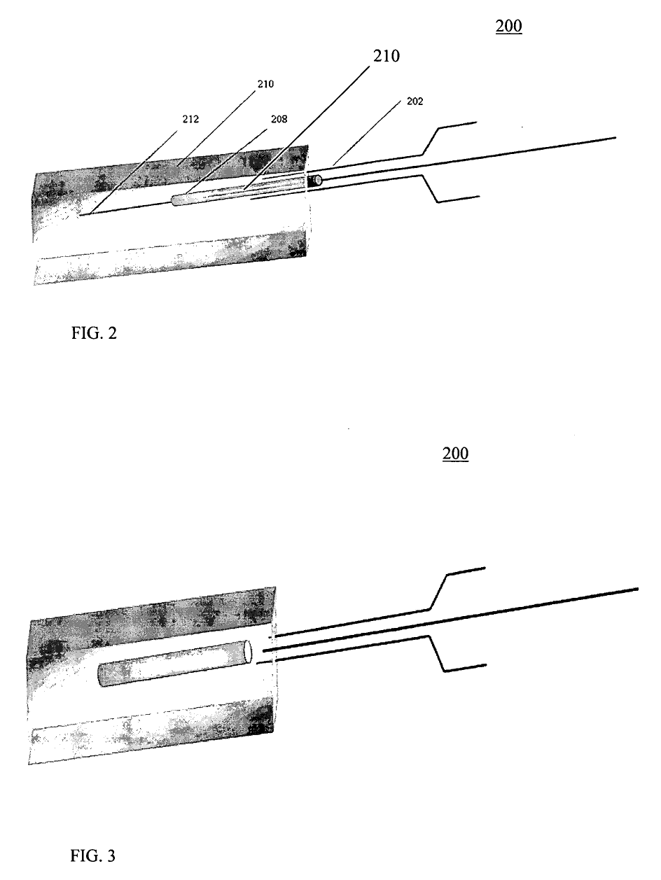

FIGS. 2-3 show an exemplary catheter 200 for providing a first approach to removing skin wrinkles. FIG. 2 shows the catheter 200 being inserted underneath the user's skin, while FIG. 3 shows the catheter 200 being removed. Catheter 200 includes sharp point 202 (insertion needle) for puncturing the skin and the underlying sub-coetaneous tissue. Catheter 200 can include a guide wire which permits the lower portion of catheter 200 including insertion needle 202 and balloon 208 to be bent to any of various selected angular positions. Thus, catheter 200, with balloon 208 deflated, may be positioned in a first position within a wrinkled skin region 210, and then inflated. Afterward, the gel may be optionally irradiated by microwave energy to stretch the skin as prescribed. Thereafter, catheter 200 may be partially withdrawn, and then, with the aid of guide wire 212, be successively repositioned in a second and third implant or balloon in the patient, and then inflated prior to being irradiated by microwave energy to form respective second and third implant to stretch the skin to reduce wrinkles.

In FIG. 3, the needle 202 is removed after deployment of a water absorbable implant or balloon 208 using the needle 202. The implant or balloon 208 is enlarged due to water absorbed from the surrounding tissue. The implant is soft and compliant to conform to the contour of the tissue. Moreover, the implant 208 fills the empty spaces causing wrinkle from the absence of collagen.

In another embodiment, two integrated-structure balloon catheters can be used for treating a relatively large-volume region such as for breast augmentation. The sharp point insertion needle of the catheter is used to puncture the skin and the underlying sub-coetaneous tissue at a position that places balloon in its deflated state within or in the vicinity of the wrinkled skin of the patient being treated and another sharp point insertion needle is used to puncture skin and the underlying sub-cutaneous tissue at a position that places a second balloon in its deflated state in a spatially spaced second position within or in the vicinity of area being augmented. The balloons are then inflated as discussed above.

### EXAMPLES

### Example 1

Formulate a monomer composition that will absorb water at a 5:1 ratio weight to weight

Make up the monomer composition to contain 10% DI water, wt/wt

Polymerize in a water bath at 65 C for 2 hours

Pour the polymer solution into a dialysis bag, place the bag into a large beaker containing DI water continuously flush the water in the beaker at a constant slow rate for 2 hours to rid the polymer of any low molecular oligomers.

Inject the 90:10 polymer solution into an implantable semi-porous, compliant balloon

The balloon containing the polymer solution may be implanted into intramuscular and/or sub-dermal tissue

At equilibrium, the polymer inside the balloon absorbed water to augmented surface skin wrinkle

### Example 2

Formulate a monomer composition that will absorb water at 5:1 ration, wt:wt, with no cross-linking

Inject the monomer mixture into a mold

The mold is the shape of the implant and is generally cylindrical, which may be at any length, and any diameter

Inject the monomer mix into the mold

The mold should be designed well to have proper vents so that no bubbles are created as the result of gaseous decomposition of the initiator and the polymerization reaction. Furthermore, the removal of the product should be relatively easy so that it will not break or crack

Polymerize the monomer mix using heat or UV accordingly depending on if the mold enclosure is better suited for UV or heat. UV generally is better because it does not out-gas as much as heat does

Formulate another monomer mixture with cross linking monomer at 1% by weight.

Remove the product from the mold and dip it into the cross linking monomer mixture. Vary the time that each product is allow to soak in the cross linking monomer mixture as this will affect the physical properties of the product.

Polymerize the product and prepare for use

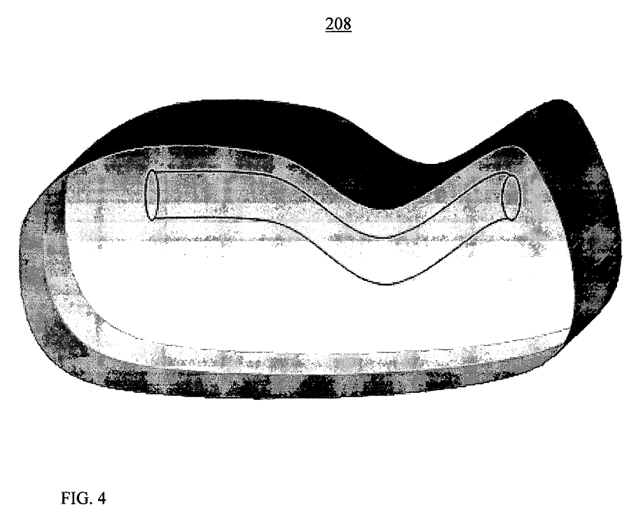

FIG. 4 shows an exemplary operation of an implant 208. The implant 208 has absorbed water, became compliant, and conformed to the general contour of the surrounding tissue. The advantage of the implant is its compliance both in stress and relaxation properties. These properties are matched to the surrounding tissue in low frequencies so that it will feel natural to the patient and others that the patient comes in contact with. The matched physical properties at low frequencies are in the polymer by design. Factors affecting the physical properties of the implant that can be adjusted during manufacturing include:
The cross linking density
The length of the cross-linker molecule, for example: 1,4 BDDMA (4 carbons, di-functional); 1,6 HDDMA (6 carbons, di-functional)
PEG-450-DMA (HW, di-functional)
Allyl methacrylate (MW, multi-functional)
The number of times that the product is repeatedly soaked in the monomer mixture as well the composition of the cross linker molecule

The system also provides improved methods of delivering sustained therapeutic dosages of medicines for extended periods. This would be more convenient to patients and reduce occurrences of missed doses. In one embodiment where the implant provides injectable medicines, the implant with drugs contained therein can maintain therapeutic levels for weeks or longer. Zero-order kinetics, wherein blood levels of drugs would remain constant throughout the delivery period. This ideal delivery is particularly important in certain classes of medicines intended, for example, for antibiotic delivery, heart and blood pressure maintenance, pain control, and antidepressants.

The implant properties can be adjusted by varying the above factors, among others. It will be appreciated by those skilled in the art that while the invention has been described above in connection with particular embodiments and examples, the invention is not necessarily so limited, and that numerous other embodiments, examples, uses, modifications and departures from the embodiments, examples and uses are intended to be encompassed by the claims attached hereto. The entire disclosure of each patent and publication cited herein is incorporated by reference, as if each such patent or publication were individually incorporated by reference herein.

What is claimed is:

1. A wrinkle removing implant, comprising:
a shell portion having one or more compartments, said shell portion implantable underneath an epidermal layer; and
a core having a limited flow gel embedded inside the shell portion.

2. The implant of claim 1, wherein the shell portion is cured after delivery under the epidermal layer.

3. The implant of claim 1, wherein the shell portion comprises one of: a balloon, an elastomeric tube, an elastomeric shell.

4. The implant of claim 1, wherein the gel is a biocompatible gel encapsulated within the shell portion.

5. The implant of claim 2, comprising a second tube or shell to cover the shell portion.

6. The implant of claim 4, wherein the second tube or shell keeps the gel from incorporating or adhering to the tissue for subsequent removal.

7. The implant of claim 1, wherein the compartment is filled with one of: saline, radio contrast agents, polymeric gel containing, non-radio contrast agents.

8. The implant of claim 1, wherein the gel comprises one of 99% PEG, Polyvinyl alcohol (PVA) dissolved in water.

9. The implant of claim 1, wherein the gel comprises 90% 2-hydroxy ethylmethacrylate, 9% butyl methacrylate, 0.1%-1% multifunctional monomers, thermally sensitive initiators or UV sensitive initiator benzoin methyl ether.

10. The implant of claim 1, wherein the gel delivers one or more drugs in a zero order method.

11. The implant of claim 1, wherein the gel comprises 90% hydroxy-terminated vinyl silicone, 9% N-vinyl pyrolidone, 0.1%-1% thionyl chloride, platinum catalyst.

12. The implant of claim 1, wherein the gel comprises particles of radio opaque materials.

13. The implant of claim 1, wherein the gel comprises one of 5%-40% barium sulfate powder, 5%-40% bismuth sulfate.

14. The implant of claim 1, wherein the gel comprises one of zirconium dioxide, silver oxides, platinum oxides, tungsten oxides.

15. The implant of claim 1, comprising an anti-inflammatory compound coated on the shell portion.

16. The implant of claim 15, wherein the anti-inflammatory compound includes one of: arthrotec, asacol, auralgan, azulfidine, bextra, celestone, daypro, deltasone, diclofenac, etodolac, indocin, ketoprofen, Iodine, mobic, nabumetone, naproxen, piroxicam, ponstan, prednisone, rofecoxib, salofalk, solumedrol.

17. The implant of claim 1, comprising an antibiotic compound including one of: Amlodipine, Besylate, Amoxicillin, Amoxil, Amphotericin, Ampicillin, Augmentin, Avelox, Bactrim, Bactroban, Biaxin, Ceftriaxone, Cefzil, Cephalexin, Chloramphenicol, Cipro XR, Clostebol, Cloxacillin, Cotrim, Daraprim, Dicloxacillin, Doxycycline, Eryacne, Erythromycin, Ethambutol.

18. The implant of claim 1, wherein the shell portion comprises a biocompatible polymer with the desired modulus that matches that of biological tissue.

19. The implant of claim 1, wherein the shell portion comprises a lightly cross-linked compound.

20. The implant of claim 1, comprising a stent to deliver the gel into a desired area.
